(19) United States
(12) Patent Application Publication
(10) Pub. No.: US 2025/0072792 A1
(43) Pub. Date: Mar. 6, 2025

(54) SYSTEM AND METHOD FOR MANAGING BLOOD GLUCOSE LEVEL

(71) Applicant: Winston R. Jeshuran, Macon, GA (US)

(72) Inventor: Winston R. Jeshuran, Macon, GA (US)

(73) Assignee: Luckstone Holdings, LLC, Macon, GA (US)

(21) Appl. No.: 18/238,551

(22) Filed: Aug. 28, 2023

(51) Int. Cl. A61B 5/145; A61B 5/00

(52) U.S. Cl. CPC A61B 5/14532; A61B 5/683; A61B 5/6885; A61B 5/746; A61B 5/7465; A61B 5/7475

(57) ABSTRACT

The present invention discloses a system and method for managing blood glucose level. The system comprises a computing device in communication with a continuous glucose monitoring (CGM) device attached to a user and a wearable device configured to be worn by the user. The computing device is configured to receive the blood glucose level of the user from the continuous glucose monitoring device, or its associated app. The computing device is further configured to enable the user to set one or more predetermined ranges of blood glucose level and set an alert for each predefined range. The computing device is further configured to determine whether the blood glucose level is within one or more predetermined ranges. The computing device is further configured to determine at least one alert corresponding to the blood glucose level and enable the wearable device to provide the determined alert until the user disables the alert.

### TECHNICAL FIELD

The present invention generally relates to disease management. More specifically, the present invention relates to a health monitoring and an alert system, and method for managing blood glucose level.

### BACKGROUND

Health conditions, for example, diabetes, require to regularly monitor the patient, make necessary adjustments to treatment plans, and ensure that the conditions are managed effectively. The management of diabetes includes the measurement of glucose and administration of a medication, such as insulin, if the individual's blood sugar is too high. In the case of a low blood sugar, the individual must take some form of oral glucose, or in the extreme case, take glucagon. Elevated blood glucose levels are, in general, less life threatening when compared to low blood glucose levels. As insulin pumps and CGMs have become more prevalent with the goal of tightly controlling blood glucose levels, there has been an increase in low blood glucose events. The average person is at a high risk of a seizure at a glucose level of 35 mg/dL, with a risk of death in this range and lower. People have normal mental function at 80 mg/dL or higher. As the individual's blood glucose drops below 70 mg/dL they experience decreased cognitive function.

If the blood glucose level of the individual falls below the normal range, the individual user will begin to lose cognitive function. The loss of cognitive function will result in the user being unable to properly correct the low blood glucose level as he/she would become incoherent. As the blood glucose level drops, the individual will progressively have decreased mental function and decreased ability to be woken. If the user is roused prior to the time of this decrease in cognitive function, then the user will be able to appropriately, correct the extremely low glucose level and therefore prevent the risk of seizure and death. As the individual has decreased cognitive function the stimulus for the individual to react must increase. Initially, a vibration may work as the individual has decreasing cognitive function. Increasing the stimulus to an audible alarm of increasing volume may work at a lower blood glucose level. There is a point when neither the vibratory nor the audible stimulus will effectively alert the individual. When this occurs, there is a high risk of the individual becoming obtunded and eventually having a seizure or dying. This is the time when an electrical stimulus of increasing voltage would be the only intervention that may rouse the individual to take action.

Few existing patent applications attempt to address the problems cited in the background as prior art over the presently disclosed subject matter and are explained as follows. US20170031449 of Peter Karsten et al. entitled “wearable device” discloses a system comprising a wearable wristband configured to be worn by a user, a control unit within the wristband, and a sensor configured to detect at least one gesture made by the user. The gesture indicating instructions are performed by the control unit. The control unit is configured to translate the at least one gesture into a specific command for an action to occur within the wristband system. The system is configured to make recommendations for a user based on the user's real-time physical, social, and biotelemetric activity. The system could be configured to provide alerts.

US20200323497 of Eli Reihman et al. entitled “Devices used in connection with continuous analyte monitoring that provide the user with one or more notifications, and related methods” discloses devices and methods for providing a user with alerts regarding the blood glucose level. The alerts may take different forms, such as an output to a display, a speaker, a vibration module, a shock module, etc. The alerts provide the user with sufficient information to take appropriate action, but the devices may be of limited functionality to enhance their compactness, discreetness, wearability, etc., while also lowering their cost to manufacture. However, improvements are desired for health monitoring devices and systems that provide one or more of effective health data collection, and monitoring to alert the users.

Therefore, there is a need for a health monitoring and alert system and method for managing low and even high/extremely high blood glucose levels.

### SUMMARY

The present invention discloses a system and method for managing blood glucose level. The system is configured to monitor the blood glucose level and provide alerts to the user regarding the blood glucose level.

The system comprises a computing device in communication with a continuous glucose monitoring device attached to a user, a first user device associated with a user, at least one second user device associated with a caretaker and a wearable device configured to be worn by the user. The continuous monitoring device is configured to continuously monitor a blood glucose level of the user. The first user device, the second user device and the wearable device are in communication with the computing device via a network. The wearable device further comprises a secure attachment assembly to attach the wearable device to the user.

The computing device is configured to receive the blood glucose level of the user from the continuous glucose monitoring device. The computing device is further configured to enable the user to set one or more predetermined ranges of blood glucose level and set an alert for each predefined range. The computing device is further configured to determine whether the blood glucose level is within one or more predetermined ranges. The computing device is further configured to determine at least one alert corresponding to the blood glucose level and enable the wearable device to provide the determined alert until the user disables the alert. The alerts include a first alert, a second alert and a third alert. In one embodiment, the first alert comprises of a vibratory and/or light-based alert, the second alert comprises of an audio and/or light-based alert and the third alert comprises electric shock of varying voltages.

The computing device is further configured to provide one or more tasks to the user to disable the alert. The tasks include entering a code, answering a question, and selecting one or more desired images from a plurality of images. The user is enabled to execute the task using the first user device. The task further includes pressing one or more buttons of the wearable device in a predefined sequence. The computing device is further configured to broadcast the blood glucose level to one or more caretakers of the user. The computing device is further configured to enable the caretaker to control the wearable device to provide at least one alert to the user.

The wearable device is configured to enable the user to set a range of intensity for the alert and deliver the alert at a predefined range of intensity. The wearable device comprises at least one detection sensor to determine whether the wearable device is attached to the user's body. The detection sensor includes at least one, but may have more than one sensor including a pressure sensor, an electrical sensor and/or a light sensor. The computing device is further configured to enable the wearable device to increase the intensity of the alert for every predefined time until the user disables the alert. The computing device is further configured to enable the wearable device to change a type of alert progressively from the first alert to the third alert for every predefined time or predefined input from the computing device, until the user disables the alert.

In one embodiment, a method for managing blood glucose level is disclosed. At one step, a system comprising a computing device in communication with a continuous glucose monitoring device attached to a user, a first user device associated with a user, at least one second user device associated with a caretaker and a wearable device configured to be worn by the user is provided. The continuous monitoring device is configured to continuously monitor the blood glucose level of the user. The first user device, the second user device and the wearable device are in communication with the computing device via a network. The wearable device further comprises a secure attachment assembly to attach the wearable device to the user.

At another step, the computing device is configured to receive the blood glucose level of the user from the continuous glucose monitoring device. At yet another step, the computing device is further configured to enable the user to set one or more predetermined ranges of blood glucose level and set an alert for each predefined range. At yet another step, the computing device is further configured to determine whether the blood glucose level is within one or more predetermined ranges.

At yet another step, the computing device is further configured to determine at least one alert corresponding to the blood glucose level and enable the wearable device to provide the determined alert until the user disables the alert. The alerts include a first alert, a second alert and a third alert. In one embodiment, the first alert comprises a vibratory alert and/or a light-based alert, the second alert comprises audio and/or a light-based alert and the third alert comprises electric shock of variable voltages.

At yet another step, the computing device is further configured to provide one or more tasks to the user to disable the alert. The tasks include entering a code, answering a question, and selecting one or more desired images from a plurality of images. The user is enabled to execute the task using the first user device. The task further includes pressing one or more buttons of the wearable device in a predefined sequence. At yet another step, the computing device is further configured to broadcast the blood glucose level to one or more caretakers of the user. At yet another step, the computing device is further configured to enable the caretaker to control the wearable device to provide at least one alert to the user.

At yet another step, the wearable device is configured to enable the user to set a range of intensity for the alert and deliver the alert at a predefined range of intensity. The wearable device comprises at least one detection sensor to determine whether the wearable device is attached to the user's body. The detection sensor includes at least one of the following: a pressure sensor, an electrical sensor and/or a light sensor. At yet another step, the computing device is further configured to enable the wearable device to increase the intensity of the alert for every predefined time until the user disables the alert. At yet another step, the computing device is further configured to enable the wearable device to change a type of alert progressively from the first alert to the third alert for every predefined time or predetermined range, until the user disables the alert.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 exemplarily illustrates an environment of a system for managing blood glucose level, according to an embodiment of the present invention.

FIG. 2 exemplarily illustrates a flowchart of a method for managing blood glucose level, according to an embodiment of the present invention.

### DETAILED DESCRIPTION OF EXAMPLE EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

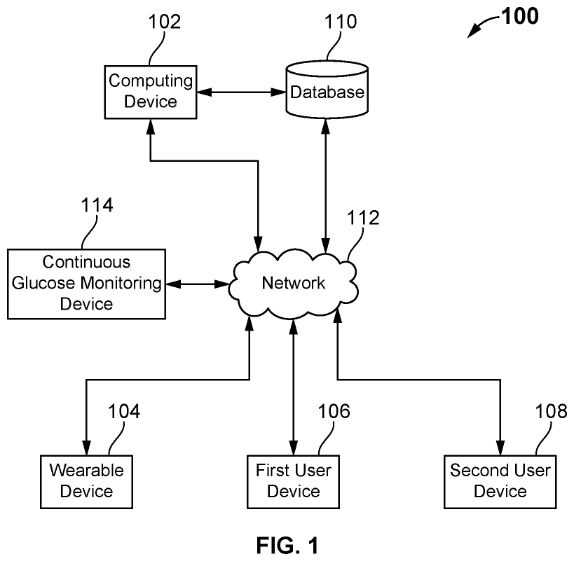

Referring to FIG. 1, an environment 100 of a system for managing blood glucose level comprises a computing device 102, a wearable device 104, a first user device 106, a second user device 108 and a database 110. The wearable device 104, at least one first user device 106, at least one second user device 108 and the database 110 are in communication the computing device 102 via a network 112.

The wearable device 104 is adapted to be worn by the user. The wearable device 104 comprises a secure attachment assembly for attachment of the device 104 to the user. The secure attachment assembly is configured to ensure that the wearable device 104 stays attached to the user during his/her sleep or while performing any other actions. In one embodiment, the secure attachment assembly comprises a strap. The strap is affixed to the wearable device 104. The strap could be attached to the user's body. In an example, the strap could be worn on the wrist, upper arm, thigh, lower leg, or ankle. In another embodiment, the wearable device 104 could be affixed to the body via an adhesive without the use of a strap. The wearable device 104 is configured to deliver one or more alerts to the user. The alert includes, but not limited to, audible alert, vibratory alert and electric shock.

The wearable device 104 comprises one or more sub components to produce alerts. In one embodiment, at least one subcomponent is configured to produce audible alert, at least one subcomponent is configured to produce vibratory alert, at least one subcomponent is configured to produce light-based alert, and at least one subcomponent is configured to produce electric shock. All of the aforementioned subcomponents will have a range therefore and could be individualized for the user.

The wearable device 104 comprises at least one detection sensor. The detection sensor is configured to determine whether the wearable device 104 is attached to the user body. The detection sensor or multiple sensors, include, but are not limited to, a pressure sensor, an electrical sensor, and/or a light sensor.

The computing device 102 is in communication with a continuous glucose monitoring device 114 attached to a user. The CGM device 114 is configured to continuously monitor a blood glucose level of the user. In one embodiment, the CGM device 114 and the wearable device 104 could be provided as an integral unit. The CGM device 114 comprises at least one analyte sensor to provide a data stream that is representative of the concentration of the analyte in the user. The analyte correlates directly to the user's blood glucose level. This data is then interpreted to determine the blood glucose level of the user.

The computing device 102 is configured to receive the blood glucose level of the user from the CGM device 114. The computing device 102 is a server. The computing device 102 could be any suitable server(s) for storing information, data, programs, and/or any other suitable content. In an example, the computing device 102 is at least one of a general or special purpose computer. The computing device 102 operates as a single computer, which could be a hardware and/or software server, a workstation, a desktop, a laptop, a tablet, a mobile phone, a mainframe, a supercomputer, a server farm, and so forth. Although the computing device 102 is illustrated as a single device, the functions performed by computing device 102 could be performed using any suitable number of computing devices.

The first user device 106 is associated with a user and the second user device 108 is associated with a caretaker. The first user device 106 and the second user device 108 are also generally referred as user device (106, 108). The user device (106, 108) is a computing device configured to provide access to the service provided by the server or the computing device 102. The user device (106, 108) is configured to provide an interface to access the services provided by the server (106, 108). The interface, for example, is an application that allows the user device (106, 108) to wirelessly connect with the server 102 via the network 112. The user device (106, 108) may be, for example, a desktop computer, a laptop computer, a mobile phone, a personal digital assistant, and the like.

The network 112 generally represents one or more interconnected networks, over which the user device (106, 108) and the computing device 102 could communicate with each other. The network 112 may include packet-based wide area networks (such as the Internet), local area networks (LAN), private networks, wireless networks, satellite networks, cellular networks, paging networks, and the like. The network 112 may include Bluetooth® connection and other similar forms of short distance communication, as the phone, the continuous glucose monitor (CGM), and the devices may be communicating through this spectrum. A person skilled in the art will recognize that the network 112 may also be a combination of more than one type of network. For example, the network 112 may be a combination of a LAN and the Internet. In addition, the network 112 may be implemented as a wired network or a wireless network or a combination thereof.

The database 110 is accessible by the computing device 102. In an example, the database 110 resides in the computing device 102. In another example, the database 110 resides separately from the computing device 102. Regardless of location, the database 110 comprises a memory to store and organize data for use by the computing device 102.

The computing device 102 comprises at least one processor and at least one memory. The memory is configured to store one or more program modules. The program modules are executed by the processor. The computing device 102 is configured to receive the blood glucose level of the user from the continuous glucose monitoring device 114. The computing device 102 is configured to enable the user to set one or more predetermined ranges of blood glucose level. The computing device 102 is configured to enable the user to set an alert for each predefined range. The wearable device 104 and the computing device 102 are configured to enable the user to set a range of intensity of the alert and deliver the alert at a predefined range of intensity. In an embodiment, the system enables the user undergo a testing to determine the levels for the initial electrical stimulation to predefine the intensity of alert.

The computing device 102 is configured to determine whether the blood glucose level is within one or more predetermined ranges. The computing device 102 is further configured to determine at least one alert corresponding to the blood glucose level and enable the wearable device 104 to provide the determined alert until the user disables the alert. The alerts include a first alert, a second alert and a third alert. The first alert comprises vibratory alert, the second alert comprises audio and light-based alert and the third alert comprises electric shock, however the alerts may be in any order predetermined by the user.

The computing device 102 is configured to enable the wearable device 104 to change a type of alert progressively from the first alert to the third alert for every predefined time until the user disables the alert. For example, if the user does not respond to vibration, sound or light-based alert, the system is configured to provide an electric shock or series of shocks of varying intensities.

The computing device 102 is configured to enable the wearable device 104 to increase the intensity of the alert for every predefined time until the user disables the alert. For example, if the user does not disable the alert in 30 seconds, the intensity of electric simulation is increased to the range set by the user until the user wakens and disarms the system. Thereby, the present invention ensures that the user takes the oral glucose or medication and thereby manages the blood glucose level.

Further, the detection sensor of the wearable device 104 is configured to determine whether the wearable device 104 is attached to the user's body. The detection sensor closes a loop between the computing device 102 and the CGM device 114. The detection sensor is to assure that the user is in contact with the wearable device 104, and that wearable device is able to provide the aforementioned alerts to the user.

The computing device 102 is configured to broadcast the blood glucose level to one or more caretakers of the user. The computing device 102 is configured to enable the caretaker to control the wearable device 104 to provide at least one alert to the user. The computing device 102 enables the caretaker to control the wearable device 104 via the second user device 108.

For example, the user could enable the wearable device 104 to deliver simulation or alert at a blood sugar level of less than 60. If the blood sugar level reaches 60, the wearable device 104 is configured to provide vibration or noise. If the alert is not turned off within a predefined time, the wearable device 104 is configured to provide electrical shock. In addition, if the blood sugar drops below 45, the user could enable the wearable device 104 to provide initial stimulation with electrical shock rather than beginning with a vibration or sound as that is a critical low level). The wearable device 104 is configured to activate a vibration, and/or sound initially in order to alert the user of a low blood sugar. If the user does not respond to the alert by either correcting the blood sugar, and confirming with the computing device 102 via the first user device 106, then the wearable device 104 is configured to deliver a noxious electrical shock. The wearable device 104 is configured to deliver multiple levels of shock. Initially, wearable device 104 is configured to provide the electrical shock at a low voltage. However, the wearable device 104 is configured to increase the voltage if the user does not turn off or disable the alert.

After the wearable device 104 has been activated to deliver an alert to the user, the progression or escalation of stimulation could be turned off by performing the tasks provided by the computing device 102. The computing device 102 is configured to provide the tasks to the first user via the first user device 106. For example, the task includes, but not limited to, entering a code, answering a question, and selecting one or more desired images from a plurality of images. This function assures that the user is not able to push a single button, turn off the alert, and then in essence disregard the alarm and not follow through on the corrective measures. This would put the user at risk progressive loss of cognitive function, a seizure or ultimately death. The user must complete the task or function in order to disarm the alert. In addition to the user interacting with the wearable device 104, the computing device 102 shares the blood glucose level of the user to their caretakers via the second user device 108. Thereby, the caretakers could receive live data of the user's blood sugar throughout day and night. The live data sharing is configured to act as a backup in case the user does not recognize their blood sugar levels. The computing device 102 is configured to enable the caretakers to deliver alert, for example, electric shock via the wearable device 104 to alert the user to take the corrective measures. Thereby, the present invention enables the user to manage blood glucose level.

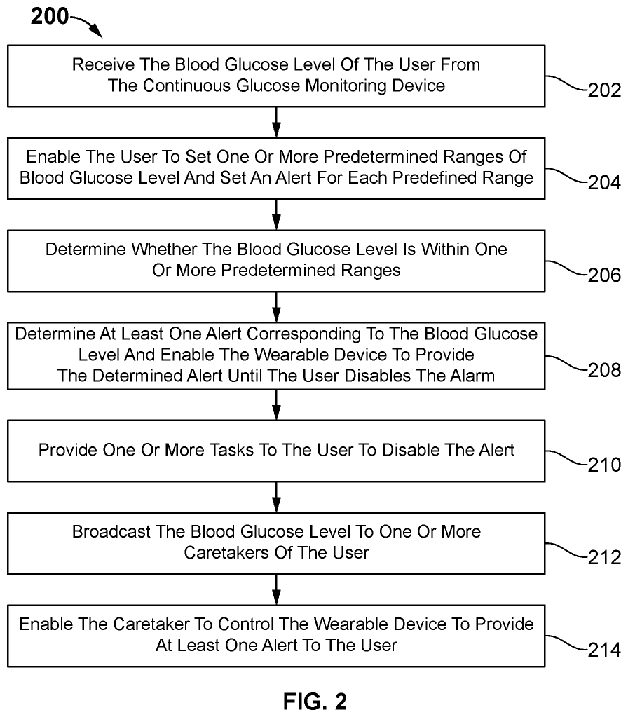

FIG. 2 exemplarily illustrates a flowchart 200 of a method for managing blood glucose level, according to an embodiment of the present invention. The method is executed in the system explained with respect to FIG. 1. The system comprises the computing device 102 in communication with the continuous glucose monitoring device 114 attached to the user, the first user device 106 associated with the user, at least one second user device 108 associated with the caretaker and the wearable device 104 worn by the user. The continuous monitoring device 114 is configured to continuously monitor a blood glucose level of the user. The first user device 106, the second user device 108 and the wearable device 104 are in communication with the computing device 102 via the network and/or Bluetooth or similar wireless communication. 112. The wearable device 104 further comprises a secure attachment assembly to attach the wearable device 104 to the user.

At step 202, the computing device 102 is configured to receive the blood glucose level of the user from the continuous glucose monitoring device 114.

At step 204, the computing device 102 is further configured to enable the user to set one or more predetermined ranges of blood glucose level and set an alert for each predefined range.

At step 206, the computing device 102 is further configured to determine whether the blood glucose level is within one or more predetermined ranges.

At step 208, the computing device 102 is further configured to determine at least one alert corresponding to the blood glucose level and enable the wearable device 104 to provide the determined alert until the user disables the alert. The alerts include a first alert, a second alert and a third alert. In one embodiment, the first alert comprises vibratory, the second alert comprises audio and light-based alert and the third alert comprises electric shock of variable voltages.

At step 210, the computing device 102 is further configured to provide one or more tasks to the user to disable the alert. The tasks include entering a code, answering a question, and selecting one or more desired images from a plurality of images. The user is enabled to execute the task using the first user device 106. The task further includes pressing one or more buttons of the wearable device 104 in a predefined sequence.

At step 212, the computing device 102 is further configured to broadcast the blood glucose level to one or more caretakers of the user. In another scenario the CGM has its own software that allows the user to share their data with the secondary user. The secondary user then receives the users CGM data in real-time. This data will then alert the secondary user. The secondary user is then able to alert the primary user though their access to the stimulation protocol of the user device.

At step 214, the computing device 102 is further configured to enable the caretaker to control the wearable device 104 to provide at least one alert to the user.

The wearable device 104 is configured to enable the user to set a range of intensity for the alert and deliver the alert at a predefined range of intensity. The wearable device 104 comprises at least one detection sensor to determine whether the wearable device 104 is attached to the user body. The caregiver is, as well, alerted as to if the device is attached to the user's body. The detection sensor includes at least one of pressure sensor, electrical sensor and light sensor. The computing device 102 is further configured to enable the wearable device 104 to increase the intensity of the alert for every predefined time until the user disables the alert. The computing device 102 is further configured to enable the wearable device 104 to change a type of alert progressively from the first alert to the third alert for every predefined time until the user disables the alert.

Advantageously, the system of the present invention causes the wearable device 104 to begin a series of alerts via at least one of vibration, sound or light. If the user does not respond to the stimulus, then electrical pulses in the form of a shock is delivered to the user. Similar to waking a patient from general anesthesia or sedation, electrical stimulus has proven to be significantly better than visual and vibratory stimuli. The system is configured to provide multiple settings for stimulation which will begin at a predetermined level and increase to a noxious level that is safe for the user however, has the best chance of rousing, the lethargic or sleeping user.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. A system for managing blood glucose level, comprising:
a computing device in communication with a continuous glucose monitoring (CGM) device attached to a user, wherein the continuous monitoring device is configured to continuously monitor a blood glucose level of the user;
a first user device associated with a user, wherein the first user device is in communication with the computing device via a network;
at least one second user device associated with a caretaker, wherein the second user device is in communication with the computing device via the network, and
a wearable device configured to be worn by the user, wherein the wearable device is in communication with the computing device via the network, wherein the computing device is configured to:
receive the blood glucose level of the user from the continuous glucose monitoring device;
enable the user to set one or more predetermined ranges of blood glucose level and set an alert and level of alert for each predefined range;
determine whether the blood glucose level is within one or more predetermined ranges;
determine at least one alert corresponding to the blood glucose level and enable the wearable device to provide the determined alert until the user disables the alert, wherein the alerts include a first alert, a second alert and a third alert, wherein the first comprises vibratory alert, the second comprises an audio and/or a light-based alert and the third alert comprises electric shock;
provide one or more tasks to the user to disable the alert;
broadcast the blood glucose level to one or more caretakers of the user, and
enable the caretaker to control the wearable device to provide at least one alert to the user.

2. The system of claim 1, wherein the wearable device is configured to enable the user to set a range of intensity for the alert and deliver the alert at a predefined range of intensity.

3. The system of claim 1, wherein the wearable device comprises at least one detection sensor to determine whether the wearable device is attached to the user body.

4. The system of claim 3, wherein the detection sensor includes at least one of pressure sensor, electrical sensor, proximity sensor and/or light sensor.

5. The system of claim 1, wherein the computing device is configured to enable the wearable device to increase the intensity of the alert for every predefined time until the user disables the alert.

6. The system of claim 1, wherein the computing device is configured to enable the wearable device to change a type of alert progressively from the first alert to the third alert for every predefined time until the user disables the alert.

7. The system of claim 1, wherein the tasks include entering a code, answering a question, and selecting one or more desired images from a plurality of images, and the user is enabled to execute the task using the first user device.

8. The system of claim 1, wherein the task includes pressing one or more buttons of the wearable device in a predefined sequence.

9. The system of claim 1, wherein the wearable device further comprises a secure attachment assembly to attach the wearable device to the user.

10. The system of claim 1, wherein the blood glucose level is broadcasted to one or more caretakers of the user, if the user shares their data with the caregiver through the CGM device.

11. A method for managing blood glucose level, comprising the steps of:
providing: a computing device in communication with a continuous glucose monitoring (CGM) device attached to a user, a first user device associated with a user, at least one second user device associated with a caretaker, and a wearable device configured to be worn by the user, wherein the first user device, the second user device and the wearable device are in communication with the computing device via a network, and wherein the continuous monitoring device is configured to continuously monitor a blood glucose level of the user;
receiving, at the computing device, the blood glucose level of the user from the continuous glucose monitoring device;
enabling, at the computing device, the user to set one or more predetermined ranges of blood glucose level and set an alert for each predefined range;
determining, at the computing device, whether the blood glucose level is within one or more predetermined ranges;
determining, at the computing device, at least one alert corresponding to the blood glucose level and enable the wearable device to provide the determined alert until the user disables the alert, wherein the alerts include a first alert, a second alert and a third alert, wherein the first alert comprises audio and light-based alert, the second alert comprises vibratory alert and the third alert comprises electric shock;
providing, at the computing device, one or more tasks to the user to disable the alert;
broadcasting, at the computing device, the blood glucose level to one or more caretakers of the user, and
enabling, at the computing device, the caretaker to control the wearable device to provide at least one alert to the user.

12. The method of claim 11, further comprises a step of: enabling, at the computing device, the user to set a range of intensity for the alert and deliver the alert at a predefined range of intensity.

13. The method of claim 11, further comprises a step of: determine, at the wearable device via a detection sensor, whether the wearable device is attached to the user body, wherein the detection sensor includes at least one of pressure sensor, proximity sensor, temperature sensor, electrical sensor and light sensor.

14. The method of claim 11, further comprises a step of: enabling the wearable device by the computing device to increase the intensity of the alert for every predefined time until the user disables the alert.

15. The method of claim 11, further comprises a step of: enabling the wearable device by the computing device to change a type of alert progressively from the first alert through the third alert for every predefined time until the user disables the alert.

16. The method of claim 11, wherein the tasks include entering a code, answering a question, and selecting one or more desired images from a plurality of images, and the user is enabled to execute the task using the first user device.

17. The method of claim 11, wherein the task includes pressing one or more buttons of the wearable device in a predefined sequence.

18. The method of claim 11, wherein the wearable device further comprises a secure attachment assembly to attach the wearable device to the user.

19. The method of claim 11, wherein the blood glucose level is broadcasted to one or more caretakers of the user, if the user shares their data with the caregiver through the CGM device.
